(19) United States
(12) Patent Application Publication
(10) Pub. No.: US 2005/0109855 A1
(43) Pub. Date: May 26, 2005

(54) Methods and apparatuses for providing a navigational array

(76) Inventor: Daniel McCombs, Memphis, TN (US)

Correspondence Address: CHIEF PATENT COUNSEL;SMITH & NEPHEW, INC., 1450 BROOKS ROAD, MEMPHIS, TN, 38116, US

(21) Appl. No.: 10/993,081

(22) Filed: Nov. 19, 2004

Related U.S. Application Data
(60) Provisional application No. 60/525,237, filed on Nov. 25, 2003.

(51) Int. Cl. G05D 23/12; A61B 5/05

(52) U.S. Cl. 236/100

(57) ABSTRACT

Methods and apparatuses for providing a navigational array for use with a computer-aided surgical navigation system. A navigational array according to an embodiment of the present invention is configured to provide a plurality of fiducial members comprising portions capable of being sensed by at least two sensors associated with the computer-aided surgical navigation system in order to determine position and orientation of the array by the system. The array can include a non-segmenting common point connecting at least some of the fiducial members, wherein at least one of the fiducial members is out of plane with the other three fiducial members, and wherein a position and orientation associated with the navigational array can be determined from sensing at least three of the fiducial members by a computer-aided surgical navigation system. The array can also include a mount adapted to support the navigational array adjacent to an object, a surgical instrument, or a joint replacement prosthesis; whereby the physical possibility for all of the fiducial members to be positioned coplanar to at least two sensors in the computer-aided surgical navigation system is precluded, and whereby when two of the fiducial members are positioned collinear to one of the sensors in the computer-aided surgical navigation system, no other of said members are positioned collinear to any other of said sensors.

### RELATED APPLICATION

This application relates to and claims the benefit on U.S. Provisional Application No. 60/525,237, filed Nov. 25, 2003 and entitled “Tetrahedral Navigation Array,” the entire contents of which are hereby expressly incorporated by this reference.

### TECHNICAL FIELD

The invention relates to computer-aided surgery, and more particularly relates to methods and apparatuses for providing a navigational array for use in a computer-aided surgery.

### BACKGROUND

Many surgical procedures require a wide array of instrumentation and other surgical items. Necessary items may include, but are not limited to: sleeves to serve as entry tools, working channels, drill guides and tissue protectors; scalpels; entry awls; guide pins; reamers; reducers; distractors; guide rods; endoscopes; arthroscopes; saws; drills; screwdrivers; awls; taps; osteotomes and wrenches. In many surgical procedures, including orthopedic procedures, it may be desirable to associate some or all of these items with a guide and/or handle incorporating a surgical reference, allowing the instrument to be used with a computer-aided surgical navigation system.

Several manufacturers currently produce computer-aided surgical navigation systems. The TREON™ and ION™ systems with FLUORONAV™ software manufactured by Medtronic Surgical Navigation Technologies, Inc. are examples of such systems. The BrainLAB VECTORVISION™ system is another example of such a surgical navigation system. Systems and processes for accomplishing computer-aided surgery are also disclosed in U.S. Ser. No. 10/084,012, filed Feb. 27, 2002 and entitled “Total Knee Arthroplasty Systems and Processes”; U.S. Ser. No. 10/084,278, filed Feb. 27, 2002 and entitled “Surgical Navigation Systems and Processes for Unicompartmental Knee Arthroplasty”; U.S. Ser. No. 10/084,291, filed Feb. 27, 2002 and entitled “Surgical Navigation Systems and Processes for High Tibial Osteotomy”; International Application No. US02/05955, filed Feb. 27, 2002 and entitled “Total Knee Arthroplasty Systems and Processes”; International Application No. US02/05956, filed Feb. 27, 2002 and entitled “Surgical Navigation Systems and Processes for Unicompartmental Knee Arthroplasty”; International Application No. US02/05783 entitled “Surgical Navigation Systems and Processes for High Tibial Osteotomy”; U.S. Ser. No. 10/364,859, filed Feb. 11, 2003 and entitled “Image Guided Fracture Reduction,” which claims priority to U.S. Ser. No. 60/355,886, filed Feb. 11, 2002 and entitled “Image Guided Fracture Reduction”; U.S. Ser. No. 60/271,818, filed Feb. 27, 2001 and entitled “Image Guided System for Arthroplasty”; and U.S. Ser. No. 10/229,372, filed Aug. 27, 2002 and entitled “Image Computer Assisted Knee Arthroplasty”, the entire contents of each of which are incorporated herein by reference as are all documents incorporated by reference therein.

These systems and processes use position and/or orientation tracking sensors such as infrared sensors acting stereoscopically or other sensors acting in conjunction with surgical references to track positions of body parts, surgery-related items such as implements, instrumentation, trial prosthetics, prosthetic components, and virtual constructs or references such as rotational axes which have been calculated and stored based on designation of bone landmarks. Processing capability such as any desired form of computer functionality, whether standalone, networked, or otherwise, takes into account the position and orientation information as to various items in the position sensing field (which may correspond generally or specifically to all or portions or more than all of the surgical field) based on sensed position and orientation of their associated surgical references, or based on stored position and/or orientation information. The processing functionality correlates this position and orientation information for each object with stored information, such as a computerized fluoroscopic imaged filed, a wire frame data file for rendering a representation of an instrument component, trial prosthesis or actual prosthesis, or a computer generated file relating to a rotational axis or other virtual construct or reference. The processing functionality then displays position and orientation of these objects on a rendering functionality, such as a screen, monitor, or otherwise. Thus, these systems or processes, by sensing the position of surgical references, can display or otherwise output useful data relating to predicted or actual position and orientation of surgical instruments, body parts, surgically related items, implants, and virtual constructs for use in navigation, assessment, and otherwise performing surgery or other operations.

Some of the surgical references used in these systems may emit or reflect infrared light that is then detected by an infrared camera. The references may be sensed actively or passively by infrared, visual, sound, magnetic, electromagnetic, x-ray or any other desired technique. An active reference emits energy, and a passive reference merely reflects energy. Some surgical references may have markers or fiducials that are traced by an infrared sensor to determine the position and orientation of the reference and thus the position and orientation of the associated instrument, item, implant component or other object to which the reference is attached.

In addition to surgical references with fixed fiducials, modular fiducials, which may be positioned independent of each other, may be used to reference points in the coordinate system. Modular fiducials may include reflective elements which may be tracked by two, sometimes more, sensors whose output may be processed in concert by associated processing functionality to geometrically calculate the position and orientation of the item to which the modular fiducial is attached. Like fixed fiducial surgical references, modular fiducials and the sensors need not be confined to the infrared spectrum—any electromagnetic, electrostatic, light, sound, radio frequency or other desired technique may be used. Similarly, modular fiducials may “actively” transmit reference information to a tracking system, as opposed to “passively” reflecting infrared or other forms of energy.

Surgical references useable with the above-identified navigation systems may be secured to any desired structure, including the above-mentioned surgical instruments and other items. The surgical references may be secured directly to the instrument or item to be referenced. However, in many instances it will not be practical or desirable to secure the surgical references to the instrument or other item. Rather, in many circumstances it will be preferred to secure the surgical references to a handle and/or a guide adapted to receive the instrument or other item. For example, drill bits and other rotating instruments cannot be tracked by securing the surgical reference directly to the rotating instrument because the reference would rotate along with the instrument. Rather, a preferred method for tracking a rotating instrument is to associate the surgical reference with the instrument or item's guide or handle.

Various arrangements and combinations of fiducials or markers, such as navigational arrays, have been implemented for use with computer-aided surgical navigation systems. Conventional navigational arrays typically include coplanar markers, wherein all of the markers are in a single plane. Use of such navigational arrays can be affected by “line of sight” problems. That is, when the angle between the plane of the array and the camera becomes acute, a marker may be obscured by other markers that are coplanar with it, resulting in limited visibility of the array. When all of the markers in the array cannot be seen in an image, locating the exact position of the marker relative to a patient's body can be difficult. When line of sight problems occur during a computer-aided surgical procedure, the position of the surgical instrument associated with the navigational array or the position of the navigational array itself must be realigned or repositioned, increasing the time and effort associated with the surgical procedure.

### SUMMARY

Various aspects and embodiments of the present invention include navigational arrays adapted to be sensed by a computer-aided surgical navigation system. Such navigational arrays can be adapted for mounting to a wide variety of surgical instruments and other items. The navigational arrays can allow particular positions and orientations of the arrays to be sensed by a computer-aided surgical navigation system. For instance, navigational arrays according to certain embodiments of the present invention may be used to locate particular positions and orientations of the array with respect to a patient's body for performance of surgical procedures, such as installation of an implant. Additionally, navigational arrays according to certain embodiments of the present invention may allow particular positions and orientations of surgical instruments and other items associated with the arrays to be registered in and tracked by a computer-aided surgical navigation system. Such systems may track the position and orientation of the surgical item by tracking the position and orientation of the surgical reference associated with the navigational array.

Navigational arrays according to certain aspects and embodiments of the present invention may include fiducial members, a common point, and a mount. In one embodiment, a plurality of fiducial members is adapted to be sensed by at least two sensors associated with a computer-aided surgical navigation system in order to determine position and orientation of the array by the system. A non-segmenting common point connects at least some of the fiducial members, wherein at least one of the fiducial members is out of plane with the other three fiducial members, and wherein a position and orientation associated with the navigational array can be determined from sensing at least three of the fiducial members by a computer-aided surgical navigation system. A mount can be adapted to support the navigational array adjacent to an object; whereby the physical possibility for all of the fiducial members to be positioned coplanar to at least two sensors in the computer-aided surgical navigation system is precluded, and whereby when two of the fiducial members are positioned collinear to one of the sensors in the computer-aided surgical navigation system, no other of said members are positioned collinear to any other of said sensors

In at least one embodiment, a navigational array can be associated with an object such as a surgical instrument or other surgically-related device.

Methods according to certain aspects and embodiments of the present invention may include a method for performing a surgical procedure using a navigational array and a computer-aided surgical navigation system. In one embodiment, a method can include mounting a navigational array adjacent to an object or surgical instrument. The navigational array in this embodiment can include a plurality of fiducial members adapted to be sensed by at least two sensors associated with the computer-aided surgical navigation system in order to determine position and orientation of the array by the system. Further, the navigational array can include a non-segmenting common point connecting at least some of the fiducial members, wherein at least one of the fiducial members is out of plane with the other three fiducial members, and wherein a position and orientation associated with the navigational array can be determined from sensing at least three of the fiducial members by a computer-aided surgical navigation system. The navigational array can also include a mount adapted to support the navigational array adjacent to an object or surgical instrument; whereby the physical possibility for all of the fiducial members to be positioned coplanar to at least two sensors in the computer-aided surgical navigation system is precluded, and whereby when two of the fiducial members are positioned collinear to one of the sensors in the computer-aided surgical navigation system, no other of said members are positioned collinear to any other of said sensors. The method can also include sensing a portion of at least three of the fiducial members by a computer-aided surgical navigation system, and determining a position associated with the object or surgical instrumentbased in part on sensing the portions of the at least three of the fiducial members.

Methods in accordance with embodiments of the invention can include a method for locating a position of a joint replacement prosthesis using a computer-aided surgical navigation system. The method can include providing a navigational array. The navigational array in this embodiment can include a plurality of fiducial members adapted to be sensed by at least two sensors associated with the computer-aided surgical navigation system in order to determine position and orientation of the array by the system. Further, the navigational array can include a non-segmenting common point connecting at least some of the fiducial members, wherein at least one of the fiducial members is out of plane with the other three fiducial members, and wherein a position and orientation associated with the navigational array can be determined from sensing at least three of the fiducial members by a computer-aided surgical navigation system. The navigational array can also include a mount adapted to support the navigational array adjacent to a joint replacement prosthesis; whereby the physical possibility for all of the fiducial members to be positioned coplanar to at least two sensors in the computer-aided surgical navigation system is precluded, and whereby when two of the fiducial members are positioned collinear to one of the sensors in the computer-aided surgical navigation system, no other of said members are positioned collinear to any other of said sensors. The method can also include mounting the navigational array adjacent to a joint replacement prosthesis, and sensing a portion of at least three of the fiducial members by a computer-aided surgical navigation system. Furthermore, the method can include determining a position associated with the joint replacement prosthesis based in part on sensing the portions of the at least three of the fiducial members, and mounting the joint replacement prosthesis to another corresponding joint replacement prosthesis for a joint replacement.

### BRIEF DESCRIPTION OF THE DRAWINGS

FIG. 1 shows a navigation array according to an embodiment of the present invention in perspective view.

FIG. 2 is a schematic view of a particular embodiment for a navigation array according to embodiments of the present invention.

FIG. 3 shows a navigational reference according to another embodiment of the present invention in perspective view mounted with respect to a patient's body.

FIG. 4 shows a navigational reference according to another embodiment of the present invention in perspective view mounted with respect to a patient's body.

FIG. 5 illustrates a flowchart of a method of use for a navigational array according to an embodiment of the present invention and a computer-aided surgical navigation system.

FIG. 6 illustrates a flowchart of a method of use for an apparatus according to an embodiment of the present invention and a computer-aided surgical navigation system.

FIG. 7 illustrates a flowchart of a method of use for an apparatus according to an embodiment of the present invention and a computer-aided surgical navigation system.

### DETAILED DESCRIPTION

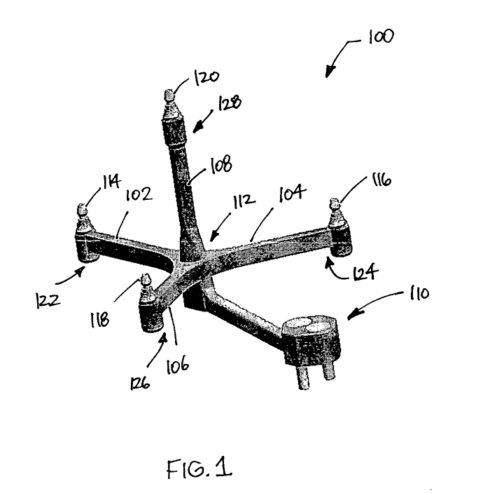

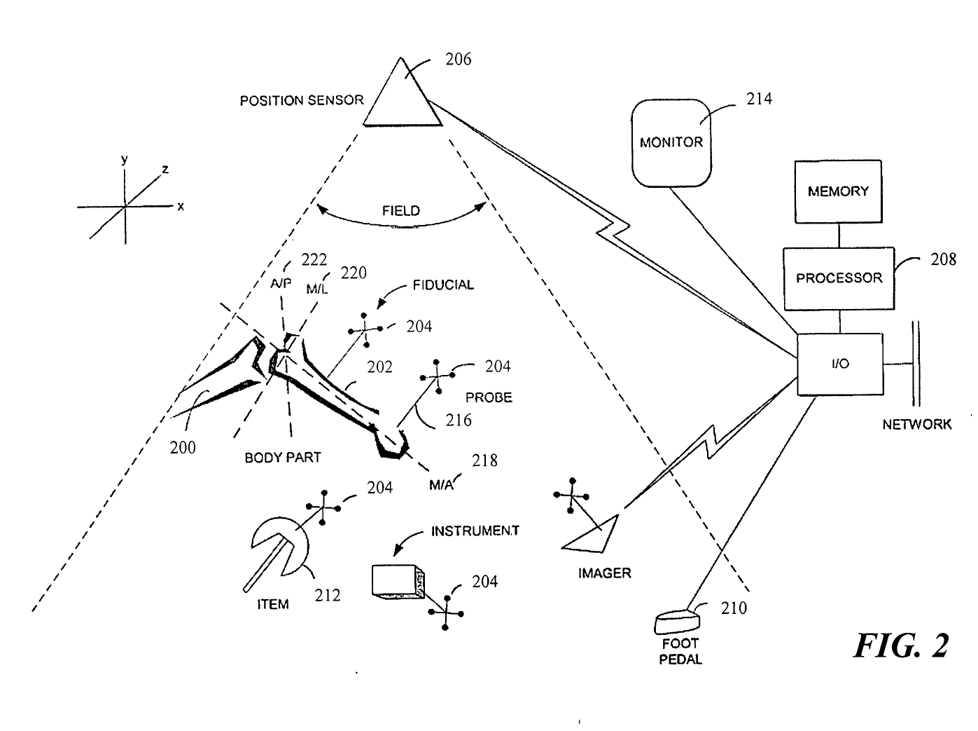

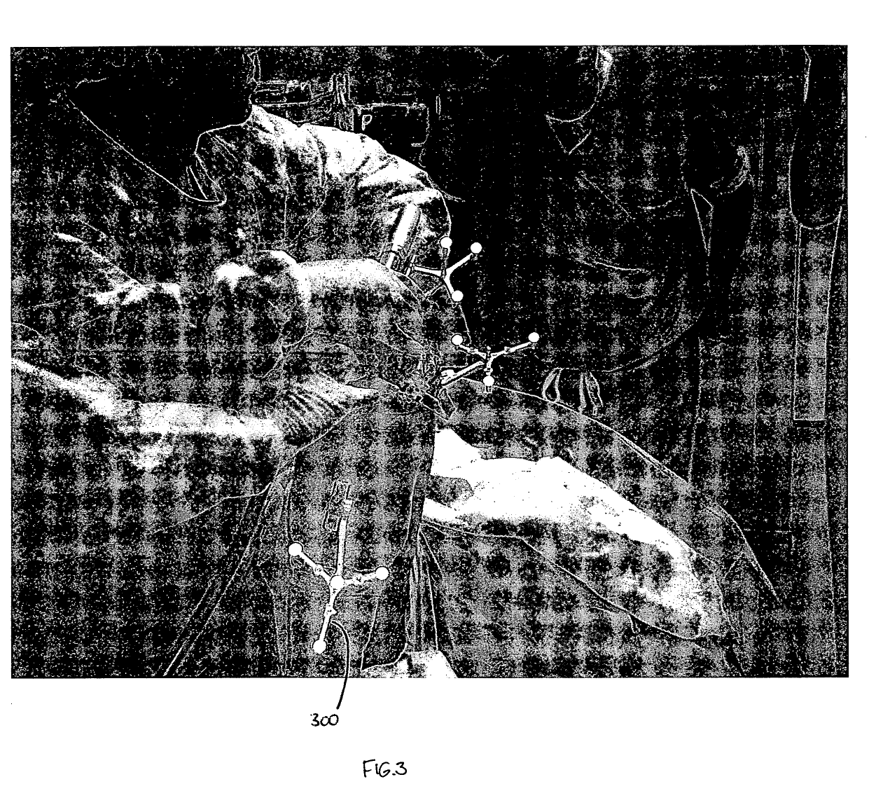

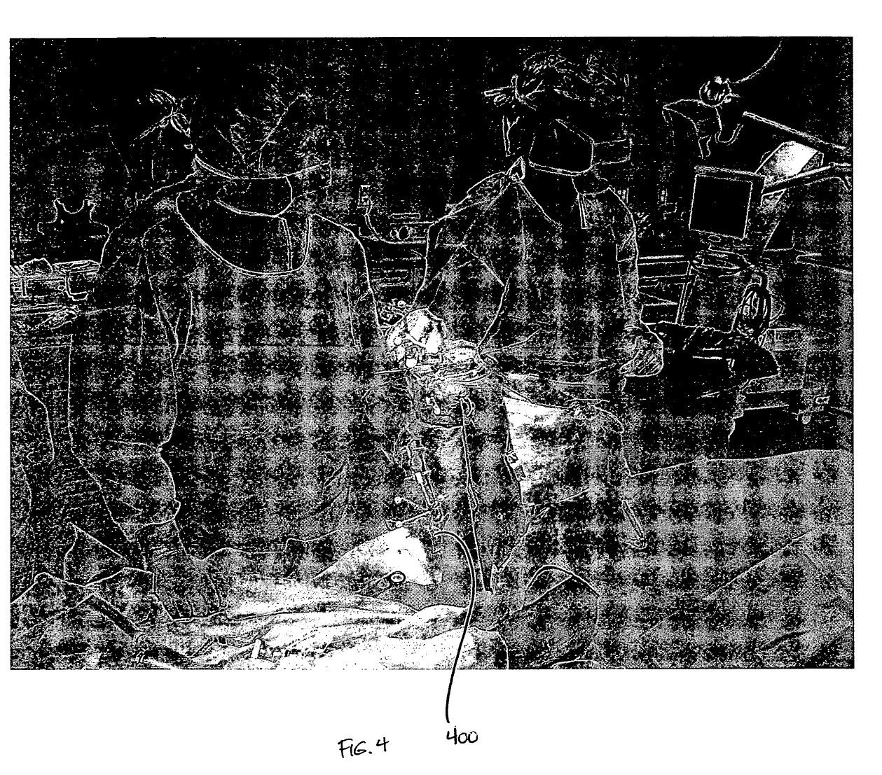

FIG. 1 shows a navigational array 100 according to a first embodiment of the present invention. A navigational array 100 can be used to define the position and orientation of various surgical instruments or other surgical items. The navigational array 100 can allow surgical instruments or items to be tracked by a computer-aided surgical navigation system, such as the 200 system shown in FIG. 2. In some embodiments, such as FIGS. 3 and 4, a navigational array 300, 400 can be used to mark exterior or interior portions of an individual to be tracked by a computer-aided surgical navigation system.

The navigational array 100 shown in FIG. 1 includes at least four fiducial members 102, 104, 106, 108, and a mount 110. Each of the fiducial members can be positioned for sensing by at least two sensors associated with a computer-aided surgical navigation system (shown in FIG. 2) in order to determine position and orientation of the array by the system. A portion of each of the four fiducial members intersects at a common point 112, while respective ends of the fiducial members 102, 104, 106, 108 extend away from the common point 112. In this example, the common point 112 connects at least some or all of the fiducial members. Furthermore in the example shown, the common point 112 is “non-segmenting” since the common point does not subdivide or segment all of the fiducial members into sub-portions. In the embodiment shown, three of the fiducial members 102, 104, 106 are arranged to form a flat Y-shaped configuration. The fourth fiducial member 108 extends from the common point 112, and is orthogonal to each of the other three fiducial members 102, 104, 106. In the example shown, each of the fiducial members 102, 104, 106, 108 are positioned orthogonal to at least one of other fiducial members 102, 104,106,108.

A respective fiducial marker, such as a marker element 114, 116, 118, 120, can mount adjacent to an opposing end 122, 124, 126, 128 of each of the fiducial members 102, 104, 106, 108. Each fiducial marker can include a conventional reflective or radiopaque material, or other material capable of being sensed by a computer-aided surgical navigation system. For example, a fiducial marker can be sensed by a computer-aided surgical navigation system using at least one of the following: infrared, sound, visual, magnetic, electromagnetic, and x-ray.

In one embodiment, a fiducial marker can be integrated with each fiducial member. For example, a fiducial member and fiducial marker can be manufactured as a single, integrated piece, wherein a portion of each fiducial member is capable of being sensed by a computer-aided surgical navigation system.

The embodiment shown in FIG. 1 provides a navigational array with at least one fiducial marker out of plane with the other three fiducial markers. That is, marker element 120 is in a separate plane from the plane formed by marker elements 114, 116, and 118. This particular arrangement for a navigational array can be used by a computer-aided surgical navigation system to locate the position and orientation of the navigational array by defining at least three fiducial markers at all times, even if one fiducial marker obscures the view of another fiducial marker from at least one view or vantage point. Additional fiducial markers or marker elements, and corresponding axial members, can be used to further reduce the sensitivity of the navigational array to any “line of sight” problems. A navigational array according to embodiments of the present invention can preclude the physical possibility for all of the fiducial members to be positioned coplanar to at least two sensors in a computer-aided surgical navigation system. Furthermore, when at least two of the fiducial members are positioned collinear to one of the sensors in the computer-aided surgical navigation system, no other of said members are positioned collinear to any other of said sensors.

The present invention is not limited to the “tetrahedral” orientation of fiducial members and markers shown in FIG. 1, and can include other orientations in accordance with embodiments of the invention. Other embodiments of a navigational array can include fewer or greater numbers of fiducial members and/or fiducial markers in accordance with embodiments of the invention. Furthermore, other configurations, shapes, and lengths of fiducial members and/or fiducial markers can exist in accordance with embodiments of the invention. In other embodiments, fiducial markers and marker elements can have different configurations than those shown in FIG. 1, such as a geometric shape, a sphere, a block, or a plate. Furthermore, in other embodiments, different geometric shapes can be used for each fiducial marker and/or marker element of a navigational array.

The mount 110 shown can associate the navigational array 100 with a portion of a patient's body, a surgical instrument, or item such as a joint replacement prosthesis. In the example shown, the mount 110 includes an arm 130 and a connector 132. The arm 130 extends between the common point 112 of the navigational array 100 and the connector 132. The connector 132 shown is a two-pronged connector that can be mounted to a corresponding two-holed connector previously mounted to a portion of a patient's body, a surgical instrument, or item such as a joint replacement prosthesis. Other configurations for a mount in accordance with embodiments of the invention can exist.

In other embodiments, the mount 110 can be any suitable structure for associating the navigational array 100 with a portion of a patient's body, a surgical instrument, or item. For example, a mount 110 can include, but is not limited to, a threaded device, a mechanical-type connecting device, a magnetic-type connecting device, an electro-mechanical or electro-magnetic type connecting device, an adhesive-type connecting device, or any other suitable connecting device adapted to associate the navigational array 100 with a portion of a patient's body, a surgical instrument, or item.

In one embodiment, a mount can be formed integrally with a surgical instrument or item such as a joint replacement prosthesis, wherein the navigational array and the surgical instrument or item can be manufactured as a single piece.

FIG. 2 is a schematic view showing an environment for using a navigational array according to the present invention in a surgery on a knee, in this case a knee arthroplasty. A navigational array according to the present invention can be used to track particular locations associated with various body parts such as tibia 200 and femur 202 to which reference arrays of the sort described above in FIG. 1 may be implanted, attached, or otherwise associated physically, virtually, or otherwise. As shown and described in the embodiment shown in FIG. 1, a navigational array can include fiducial markers, such as marker elements, capable of being sensed by a computer-aided surgical navigation system. In the example shown in FIG. 2, a position sensor 206 can sense, store, process and/or output data relating to position and orientation of reference arrays 204 and thus components such as 200 and 202 to which they are attached or otherwise associated. The position sensor 206, as mentioned above, may be any sort of sensor functionality for sensing position and orientation of reference arrays 204 and therefore items with which they are associated, according to whatever desired electrical, magnetic, electromagnetic, sound, physical, radio frequency, or other active or passive technique. In one embodiment, position sensor 206 is a pair of infrared sensors disposed on the order of a meter, sometimes more, sometimes less, apart and whose output can be processed in concert to provide position and orientation information regarding navigational arrays 204.

In the embodiment shown in FIG. 2, computing functionality 208 can include processing functionality, memory functionality, input/output functionality whether on a standalone or distributed basis, via any desired standard, architecture, interface and/or network topology. In one embodiment, computing functionality 208 can be connected to a monitor on which graphics and data may be presented to the surgeon during surgery. The screen preferably has a tactile interface so that the surgeon may point and click on screen for tactile screen input in addition to or instead of, if desired, keyboard and mouse conventional interfaces. Additionally, a foot pedal 210 or other convenient interface may be coupled to functionality 208 as can any other wireless or wireline interface to allow the surgeon, nurse or other desired user to control or direct functionality 208 in order to, among other things, capture position/orientation information when certain components are oriented or aligned properly. Items 212 such as trial components, instrumentation components may be tracked in position and orientation relative to body parts 200 and 202 using one or more navigational arrays 204.

Computing functionality 208 can process, store and output on monitor 214 and otherwise various forms of data which correspond in whole or part to body parts 200 and 202 and other components for item 212. For example, body parts 200 and 202 can be shown in cross-section or at least various internal aspects of them such as bone canals and surface structure can be shown using fluoroscopic images. These images can be obtained using a C-arm attached to a navigational array 204. The body parts, for example, tibia 200 and femur 202, can also have navigational arrays 204 attached. When fluoroscopy images are obtained using the C-arm with a navigational array 204, a position/orientation sensor 206 “sees” and tracks the position of the fluoroscopy head as well as the positions and orientations of the tibia 200 and femur 202. The computer stores the fluoroscopic images with this position/orientation information, thus correlating position and orientation of the fluoroscopic image relative to the relevant body part or parts. Thus, when the tibia 200 and corresponding navigational array 204 move, the computer automatically and correspondingly senses the new position of tibia 200 in space and can correspondingly move implements, instruments, references, trials and/or implants on the monitor 214 relative to the image of tibia 200. Similarly, the image of the body part can be moved, both the body part and such items may be moved, or the on screen image otherwise presented to suit the preferences of the surgeon or others and carry out the imaging that is desired. Similarly, when an item 212, such as a stylus, cutting block, reamer, drill, saw, extramedullary rod, intramedullar rod, or any other type of item or instrument, that is being tracked moves, its image moves on monitor 214 so that the monitor shows the item 212 in proper position and orientation on monitor 214 relative to the femur 202. The item 212 can thus appear on the monitor 214 in proper or improper alignment with respect to the mechanical axis and other features of the femur 202, as if the surgeon were able to see into the body in order to navigate and position item 212 properly.

The computer functionality 208 can also store data relating to configuration, size and other properties of items 212 such as joint replacement prostheses, implements, instrumentation, trial components, implant components and other items used in surgery. When those are introduced into the field of position/orientation sensor 206, computer functionality 208 can generate and display overlain or in combination with the fluoroscopic images of the body parts 200 and 202, computer generated images of joint replacement prostheses, implements, instrumentation components, trial components, implant components and other items 212 for navigation, positioning, assessment and other uses.

Computer functionality 208 may also store and output virtual construct data based on the sensed position and orientation of items in the surgical field, such as surgical instruments. For example, monitor 214 can output a resection plane that corresponds to the resection plane defined by a cutting guide whose position and orientation is being tracked by sensors 206. In other embodiments, monitor 214 can output a cutting track based on the sensed position and orientation of a reamer. Other virtual constructs can also be output on monitor 214, and can be displayed with or without the relevant surgical instrument, based on the sensed position and orientation of any surgical instrument or other item in the surgical field to assist the surgeon or other user to plan some or all of the stages of the surgical procedure.

In some preferred embodiments of the present invention, computer functionality can output on monitor 214 the projected position and orientation of an implant component or components based on the sensed position and orientation of one or more surgical instruments associated with one or more navigational arrays 204. For example, the system may track the position and orientation of a cutting block as it is navigated with respect to a portion of a body part that will be resected. Computer functionality 208 may calculate and output on monitor 214 the projected placement of the implant in the body part based on the sensed position and orientation of the cutting block. If the surgeon or other user is dissatisfied with the projected placement of the implant, the surgeon may then reposition the cutting block to evaluate the effect on projected implant position and orientation.

Additionally, computer functionality 208 can track any point in the position/orientation sensor 206 field such as by using a designator or a probe 216. The probe also can contain or be attached to a navigational array 204. The surgeon, nurse, or other user touches the tip of probe 216 to a point such as a landmark on bone structure and actuates the foot pedal 210 or otherwise instructs the computer 208 to note the landmark position. The position/orientation sensor 206 “sees” the position and orientation of navigational array 204 “knows” where the tip of probe 216 is relative to that navigational array 204 and thus calculates and stores, and can display on monitor 214 whenever desired and in whatever form or fashion or color, the point or other position designated by probe 216 when the foot pedal 210 is hit or other command is given. Thus, probe 216 can be used to designate landmarks on bone structure in order to allow the computer 208 to store and track, relative to movement of the navigational array 204, virtual or logical information such as mechanical axis 218, medial lateral axis 220 and anterior/posterior axis 222 of femur 202, tibia 200 and other body parts in addition to any other virtual or actual construct or reference.

A navigational array according to an embodiment of the present invention such as the subject of FIG. 1, can use the so-called FluoroNAV system and software provided by Medtronic Sofamor Danek Technologies. Such systems or aspects of them are disclosed in U.S. Pat. Nos. 5,383,454; 5,871,445; 6,146,390; 6,165,81; 6,235,038 and 6,236,875, and related (under 35 U.S.C. Section 119 and/or 120) patents, which are all incorporated herein by this reference. Any other desired systems can be used as mentioned above for imaging, storage of data, tracking of body parts and items and for other purposes.

The FluoroNav system can require the use of reference frame type fiducials which have four and in some cases five elements tracked by infrared sensors for position/orientation of the fiducials and thus of the body part, implement, instrumentation, trial component, implant component, or other device or structure being tracked. Such systems can also use at least one probe 216 which the surgeon can use to select, designate, register, or otherwise make known to the system a point or points on the anatomy or other locations by placing the probe as appropriate and signaling or commanding the computer to note the location of, for instance, the tip of the probe. The FluoroNav system can also track position and orientation of a C-arm used to obtain fluoroscopic images of body parts to which fiducials have been attached for capturing and storage of fluoroscopic images keyed to position/orientation information as tracked by the sensors 206. Thus, the monitor 214 can render fluoroscopic images of bones in combination with computer generated images of virtual constructs and references together with implements, instrumentation components, trial components, implant components and other items used in connection with surgery for navigation, resection of bone, assessment and other purposes.

Various embodiments of the invention can be used with point of class-type, registration-type, and other surgical location and preparation techniques and methods. For example, in one prosthetic installation procedure, a surgeon can designate a center of rotation of a patient's femoral head for purposes of establishing the mechanical axis and other relevant constructs relating to the patient's femur according to which prosthetic components can ultimately be positioned. Such center of rotation can be established by articulating the femur within the acetabulum or a prosthesis to capture a number of samples of position and orientation information and thus in turn to allow the computer to calculate the average center of rotation. The center of rotation can be established by using a probe associated with a navigational array, and designating a number of points on the femoral head and thus allowing the computer to calculate the geometrical center or a center which corresponds to the geometry of points collected. Additionally, graphical representations such as controllably sized circles displayed on the monitor can be fitted by the surgeon to the shape of the femoral head on planar images using tactile input on screen to designate the centers according to that graphic, such as are represented by the computer as intersection of axes of the circles. Other techniques for determining, calculating or establishing points or constructs in space, whether or not corresponding to bone structure, can be used in accordance with the present invention.

In another example, a navigational array according to various embodiments of the invention can be used in designation or registration of items which will be used in surgery. Registration simply means, however it is accomplished, ensuring that the computer knows which body part, item or construct corresponds to which fiducial or fiducials, and how the position and orientation of the body part, item or construct is related to the position and orientation of its corresponding fiducial or a fiducial attached to an impactor or other other component which is in turn attached to an item. Such registration or designation can be done before or after registering bone or body parts. In one instance, a technician can designate with a probe an item such as an instrument component to which a navigational array is attached. A sensor associated with a computer-aided surgical navigational system can “see” the position and orientation of the navigational array attached to the item and also the position and orientation of the navigational array attached to the probe whose tip is touching a landmark on the item. The technician can designate onscreen or otherwise the identification of the item and then activates the foot pedal or otherwise instructs the computer to correlate the data corresponding to such identification, such as data needed to represent a particular cutting block component for a particular knee implant product, with the particularly shaped navigational array attached to the component. The computer has then stored identification, position and orientation information relating to the navigational array for the component correlated with the data such as configuration and shape data for the item so that upon registration, when the sensor can track the item and navigational array in the infrared field, the monitor can show the cutting block component moving and turning, and properly positioned and oriented relative to the body part which is also being tracked.

Similarly, the mechanical axis and other axes or constructs of body parts can also be “registered” for tracking by the system. Again, the computer-aided surgical navigational system can employ a fluoroscope to obtain images of the patient's femoral head, knee and ankle, or other body parts. The system can correlate such images with the position and orientation of the C-arm and the patient anatomy in real time as discussed above with the use of one or more navigational arrays placed on the body parts before image acquisition and which remain in position during the surgical procedure. Using these images and/or the probe, the surgeon can select and register in the computer the center of the femoral head and ankle in orthogonal views, usually anterior/posterior and lateral, on a touch screen. The surgeon can use the probe to select any desired anatomical landmarks or references at the operative site of the knee or on the skin or surgical draping over the skin, as on the ankle. These points can be registered in three dimensional space by the system and can be tracked relative to the navigational arrays on the patient anatomy which are preferably placed intraoperatively. Although registering points using actual bone structure is one preferred way to establish the axis, a cloud of points approach by which the probe is used to designate multiple points on the surface of the bone structure can be employed, as can moving the body part and tracking movement to establish a center of rotation as discussed above. Once the center of rotation for the femoral head and the condylar component have been registered, the computer can calculate, store, and render, and otherwise use data for, the mechanical axis of the femur.

In one example, a tibial mechanical axis can be established by designating points to determine the centers of the proximal and distal ends of a patient's tibia so that the mechanical axis can be calculated, stored, and subsequently used by the computer. A posterior condylar axis can also determined by designating points or as otherwise desired, as rendered on the computer generated geometric images overlain or displayed in combination with the fluoroscopic images, all of which are keyed to one or more navigational arrays being tracked by sensors associated with the computer-aided surgical navigational system.

The above methods and techniques are provided by way of example only, and other embodiments of the present invention can be used with other surgical location and preparation techniques and methods.

FIGS. 3 and 4 show a navigational array according to another embodiment of the present invention in perspective view mounted with respect to a portion of a patient's body, and capable of being tracked with a computer-aided surgical navigation system. The computer-aided surgical navigation system used to track the navigational arrays 300, 400 can be similar to the system shown in FIG. 2. In the examples shown in FIGS. 3 and 4, navigational arrays 300, 400 are rigidly attached to a patient's tibia and adjacent to the knee. Attachment of the navigational arrays 300, 400 preferably is accomplished using a structure that corresponds with the respective mounts of the navigational arrays. Such structures can preferably withstand vibration of surgical saws and other phenomenon which occur during surgery without allowing any substantial movement of the navigational arrays 300, 400 relative to the body part being tracked by the computer-aided surgical navigation system.

The fiducial markers or marker elements of the navigational arrays 300, 400 shown are capable of being tracked by sensors 206 of the computer-aided surgical navigation system. Thus, when the fiducial markers or marker elements are sensed by the computer-aided surgical navigation system, the system can determine positions associated with the navigational arrays 300, 400.

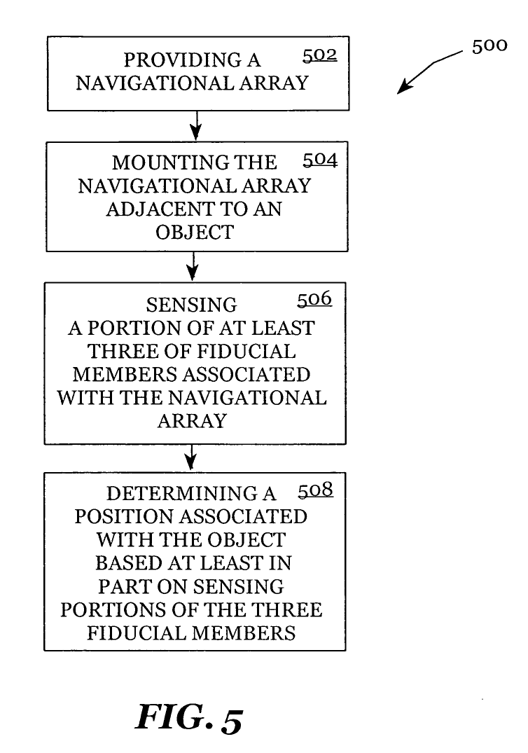

FIG. 5 illustrates a flowchart of a method 500 of use for a navigational array according to an embodiment of the present invention and a computer-aided surgical navigation system.

The method begins at block 502. At block 502, a navigational array is provided. In the embodiment shown in FIG. 5, the navigational array can be similar to the navigational array 100 shown in FIG. 1. The navigational array in this example can include a plurality of fiducial members, such as a first, second, third, and fourth fiducial member, capable of being positioned for sensing by at least two sensors associated with a computer-aided surgical navigation system (shown in FIG. 2) in order to determine position and orientation of the array by the system. Each fiducial member can include a portion adapted to be sensed by a computer-aided surgical navigation system, such as a fiducial marker or marker element. Furthermore, the navigational array can also include a non-segmenting common point connecting at least some of the fiducial members, wherein at least one of the fiducial members is out of plane with the other fiducial members, and wherein a position and orientation associated with the navigational array can be determined from sensing at least three of the fiducial members by a computer-aided surgical navigation system. Moreover, the navigational array can include a mount adapted to support the navigational array adjacent to an object.

Block 502 is followed by block 504, in which a navigational array is mounted adjacent to an object. The mount associated with the navigational array can be utilized to support the array adjacent to an object, such as a portion of a patient's body. An object in this embodiment can include at least one of the following: a patient's bone, a surgical implement, a surgical reference, a surgical trial, an implant, a cutting block, a reamer, a drill, a saw, an extramedullary rod, and an intramedullar rod.

Block 504 is followed by block 506, in which a portion of at least three of the fiducial members is sensed by the computer-aided surgical navigation system. In the embodiment shown in FIG. 5, a computer-aided surgical navigation system similar to that shown in FIG. 2, can be used to sense portions of at least three fiducial members associated with the navigational array.

Block 506 is followed by block 508, in which a position associated with the object is determined based at least in part on sensing the portions of the at least three fiducial members.

The method 500 ends at block 508. Other method elements can exist in accordance with embodiments of the invention.

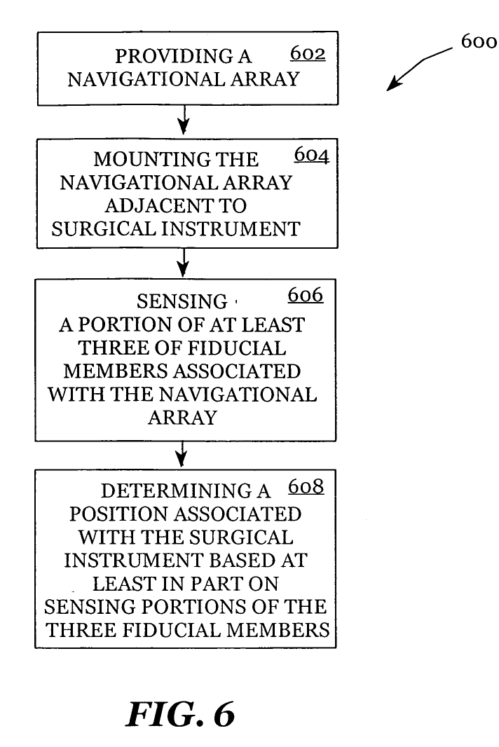

FIG. 6 illustrates a flowchart of a method 600 of use for an apparatus according to an embodiment of the present invention and a computer-aided surgical navigation system.

The method begins at block 602. At block 602, a navigational array is provided. In the embodiment shown in FIG. 6, the navigational array can be similar to the navigational array 100 shown in FIG. 1. The navigational array in this example can include a plurality of fiducial members, such as a first, second, third, and fourth fiducial member, capable of being positioned for sensing by at least two sensors associated with a computer-aided surgical navigation system (shown in FIG. 2) in order to determine position and orientation of the array by the system. Each fiducial member can include a portion adapted to be sensed by a computer-aided surgical navigation system, such as a fiducial marker or marker element. Furthermore, the navigational array can also include a non-segmenting common point connecting at least some of the fiducial members, wherein at least one of the fiducial members is out of plane with the other fiducial members, and wherein a position and orientation associated with the navigational array can be determined from sensing at least three of the fiducial members by a computer-aided surgical navigation system. Moreover, the navigational array can include a mount adapted to support the navigational array adjacent to an object.

Block 602 is followed by block 604, in which a navigational array is mounted adjacent to a surgical instrument. A surgical instrument in this embodiment can include at least one of the following: a surgical implement, a surgical reference, a surgical trial, an implant, a cutting block, a reamer, a drill, a saw, an extramedullary rod, and an intramedullar rod.

Block 604 is followed by block 606, in which a portion of at least three of the fiducial members is sensed by the computer-aided surgical navigation system. In the embodiment shown in FIG. 6, a computer-aided surgical navigation system similar to that shown in FIG. 2, can be used to sense portions of at least three fiducial members associated with the navigational array.

Block 606 is followed by block 608, in which a position associated with the apparatus is determined based at least in part on sensing the portions of the at least three fiducial members.

The method 600 ends at block 608. Other method elements can exist in accordance with embodiments of the invention.

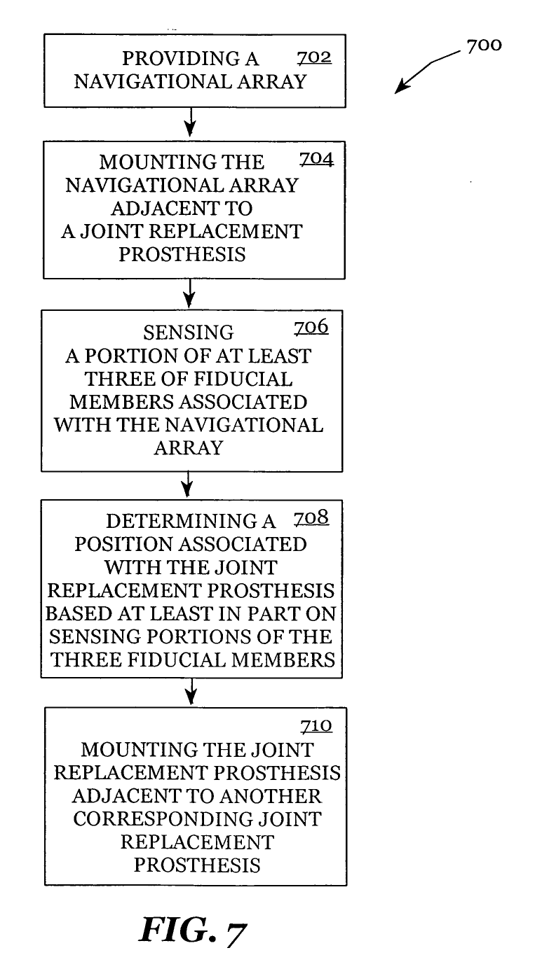

FIG. 7 illustrates a flowchart of a method 700 of use for a navigational array according to an embodiment of the present invention and a computer-aided surgical navigation system.

The method begins at block 702. At block 702, a navigational array is provided. In the embodiment shown in FIG. 7, the navigational array can be similar to the navigational array 100 shown in FIG. 1. The navigational array in this example can include a plurality of fiducial members, such as a first, second, third, and fourth fiducial member, capable of being positioned for sensing by at least two sensors associated with a computer-aided surgical navigation system (shown in FIG. 2) in order to determine position and orientation of the array by the system. Each fiducial member can include a portion adapted to be sensed by a computer-aided surgical navigation system, such as a fiducial marker or marker element. Furthermore, the navigational array can also include a non-segmenting common point connecting at least some of the fiducial members, wherein at least one of the fiducial members is out of plane with the other fiducial members, and wherein a position and orientation associated with the navigational array can be determined from sensing at least three of the fiducial members by a computer-aided surgical navigation system. Moreover, the navigational array can include a mount adapted to support the navigational array adjacent to an object.

Block 702 is followed by block 704, in which a navigational array is mounted adjacent to a joint replacement prosthesis. A joint replacement prosthesis can include, but is not limited to, a tibial component, and a femoral component.

Block 704 is followed by block 706, in which a portion of at least three of the fiducial members can be sensed by a computer-aided surgical navigation system.

Block 706 is followed by block 708, in which a position associated with the joint replacement prosthesis is determined based in part on sensing the portions of the at least three of the fiducial members.

Block 708 is followed by block 710, in which the joint replacement prosthesis is mounted to another corresponding joint replacement prosthesis for a joint replacement.

At block 710, the method 700 ends.

Changes and modifications, additions and deletions may be made to the structures and methods recited above and shown in the drawings without departing from the scope or spirit of the invention and the following claims.

1. A navigational array for use by a computer-aided surgical navigation system, wherein the navigational array can be positioned for sensing by at least two sensors associated with the computer-aided surgical navigation system in order to determine position and orientation of the array by the system, the navigational array comprising:
a plurality of fiducial members adapted to be sensed by a computer-aided surgical navigation system;
a non-segmenting common point connecting at least some of the fiducial members, wherein at least one of the fiducial members is out of plane with the other three fiducial members, and wherein a position and orientation associated with the navigational array can be determined from sensing at least three of the fiducial members by a computer-aided surgical navigation system; and
a mount adapted to support the navigational array adjacent to an object;
whereby the physical possibility for all of the fiducial members to be positioned coplanar to at least two sensors in the computer-aided surgical navigation system is precluded, and whereby when two of the fiducial members are positioned collinear to one of the sensors in the computer-aided surgical navigation system, no other of said members are positioned collinear to any other of said sensors.

2. The navigational array of claim 1, wherein at least three of the fiducial members are oriented in a flat, coplanar, Y-shaped configuration, and a fourth fiducial member is orthogonally oriented to each of the other three fiducial members.

3. The navigational array of claim 1, wherein at least a portion of each fiducial member can be sensed by the computer-aided surgical navigation system using at least one of the following: infrared, sound, visual, magnetic, electromagnetic, and x-ray.

4. The navigational array of claim 1, wherein each fiducial member comprises a respective fiducial marker.

5. The navigational array of claim 4, wherein the fiducial markers can be sensed by a computer-aided surgical navigation system using at least one of the following: infrared, sound, visual, magnetic, electromagnetic, and x-ray.

6. The navigational array of claim 4, wherein the fiducial markers can be at least one of the following: a geometric shape, a sphere, a block, and a plate.

7. The navigational array of claim 1, wherein the mount can be mounted to at least one of the following: a portion of a patient's body, a surgical instrument, a surgical item, an item associated with a surgery.

8. The navigational array of claim 1, wherein the mount comprises at least one of the following: a pronged connector, a magnet, a threaded connector, an adhesive, and a bone screw.

9. A method for performing a surgical procedure using a navigational array and a computer-aided surgical navigation system, wherein the navigational array can be positioned for sensing by at least two sensors associated with the computer-aided surgical navigation system in order to determine position and orientation of the array by the system, the method comprising:
(a) mounting a navigational array adjacent to an object, wherein the navigational array comprises:
(i) a plurality of fiducial members adapted to be sensed by the computer-aided surgical navigation system;
(ii) a non-segmenting common point connecting at least some of the fiducial members, wherein at least one of the fiducial members is out of plane with the other three fiducial members, and wherein a position and orientation associated with the navigational array can be determined from sensing at least three of the fiducial members by a computer-aided surgical navigation system;
(iii) a mount adapted to support the navigational array adjacent to an object; and
(iv) whereby the physical possibility for all of the fiducial members to be positioned coplanar to at least two sensors in the computer-aided surgical navigation system is precluded, and whereby when two of the fiducial members are positioned collinear to one of the sensors in the computer-aided surgical navigation system, no other of said members are positioned collinear to any other of said sensors;
(b) sensing a portion of at least three of the fiducial members by a computer-aided surgical navigation system; and
(c) determining a position associated with the object based in part on sensing the portions of the at least three of the fiducial members.

10. The method of claim 9, wherein at least three of the fiducial members are oriented in a flat, coplanar, Y-shaped configuration, and a fourth fiducial member is orthogonally oriented to each of the other three fiducial members.

11. The method of claim 9, wherein at least a portion of each fiducial member can be sensed by the computer-aided surgical navigation system using at least one of the following: infrared, sound, visual, magnetic, electromagnetic, and x-ray.

12. The method of claim 9, wherein each fiducial member comprises a respective fiducial marker.

13. The method of claim 12, wherein the fiducial markers can be sensed by a computer-aided surgical navigation system using at least one of the following: infrared, sound, visual, magnetic, electromagnetic, and x-ray.

14. The method of claim 12, wherein the fiducial markers can be at least one of the following: a geometric shape, a sphere, a block, and a plate.

15. The method of claim 9, wherein the mount can be mounted to at least one of the following: a portion of a patient's body, a surgical instrument, a surgical item, an item associated with a surgery.

16. The method of claim 9, wherein the mount comprises at least one of the following: a pronged connector, a magnet, a threaded connector, an adhesive, and a bone screw.

17. An apparatus for use by a computer-aided surgical navigation system, wherein the apparatus can be positioned for sensing by at least two sensors associated with the computer-aided surgical navigation system in order to determine position and orientation of the apparatus by the system, the system comprising:
(a) a surgical instrument; and
(b) a navigational array comprising:
(i) a plurality of fiducial members adapted to be sensed by the computer-aided surgical navigation system;
(ii) a non-segmenting common point connecting at least some of the fiducial members, wherein at least one of the fiducial members is out of plane with the other three fiducial members, and wherein a position and orientation associated with the navigational array can be determined from sensing at least three of the fiducial members by a computer-aided surgical navigation system;
(iii) a mount adapted to support the navigational array adjacent to the surgical instrument; and
(iv) whereby the physical possibility for all of the fiducial members to be positioned coplanar to at least two sensors in the computer-aided surgical navigation system is precluded, and whereby when two of the fiducial members are positioned collinear to one of the sensors in the computer-aided surgical navigation system, no other of said members are positioned collinear to any other of said sensors.

18. The apparatus of claim 17, wherein at least three of the fiducial members are oriented in a flat, coplanar, Y-shaped configuration, and a fourth fiducial member is orthogonally oriented to each of the other three fiducial members.

19. The apparatus of claim 17, wherein at least a portion of each fiducial member can be sensed by the computer-aided surgical navigation system using at least one of the following: infrared, sound, visual, magnetic, electromagnetic, and x-ray.

20. The apparatus of claim 17, wherein each fiducial member comprises a respective fiducial marker.

21. The apparatus of claim 20, wherein the fiducial markers can be sensed by a computer-aided surgical navigation system using at least one of the following: infrared, sound, visual, magnetic, electromagnetic, and x-ray.

22. The apparatus of claim 20, wherein the fiducial markers can be at least one of the following: a geometric shape, a sphere, a block, and a plate.

23. The apparatus of claim 17, wherein the mount can be mounted to at least one of the following: a portion of a patient's body, a surgical instrument, a surgical item, an item associated with a surgery.

24. The apparatus of claim 17, wherein the mount comprises at least one of the following: a pronged connector, a magnet, a threaded connector, an adhesive, and a bone screw.

25. The apparatus of claim 17, wherein the surgical instrument comprises at least one of the following: a surgical implement, a surgical reference, a surgical trial, an implant, a cutting block, a reamer, a drill, a saw, an extramedullary rod, and an intramedullar rod.

26. A method for performing a surgical procedure using an apparatus and a computer-aided surgical navigation system, wherein the apparatus can be positioned for sensing by at least two sensors associated with the computer-aided surgical navigation system in order to determine position and orientation of the apparatus by the system, the method comprising:
(a) mounting a navigational array adjacent to a surgical instrument, wherein the navigational array comprises:
(i) a plurality of fiducial members adapted to be sensed by the computer-aided surgical navigation system;
(ii) a non-segmenting common point connecting at least some of the fiducial members, wherein at least one of the fiducial members is out of plane with the other three fiducial members, and wherein a position and orientation associated with the navigational array can be determined from sensing at least three of the fiducial members by a computer-aided surgical navigation system;
(iii) a mount adapted to support the navigational array adjacent to the surgical instrument; and
(iv) whereby the physical possibility for all of the fiducial members to be positioned coplanar to at least two sensors in the computer-aided surgical navigation system is precluded, and whereby when two of the fiducial members are positioned collinear to one of the sensors in the computer-aided surgical navigation system, no other of said members are positioned collinear to any other of said sensors;
(b) sensing a portion of at least three of the fiducial members by a computer-aided surgical navigation system; and
(c) determining a position associated with the surgical instrument based in part on sensing the portions of the at least three of the fiducial members.

27. The method of claim 26, wherein at least three of the fiducial members are oriented in a flat, coplanar, Y-shaped configuration, and a fourth fiducial member is orthogonally oriented to each of the other three fiducial members.

28. The method of claim 26, wherein at least a portion of each fiducial member can be sensed by the computer-aided surgical navigation system using at least one of the following: infrared, sound, visual, magnetic, electromagnetic, and x-ray.

29. The method of claim 26, wherein each fiducial member comprises a respective fiducial marker.

30. The method of claim 29, wherein the fiducial markers can be sensed by a computer-aided surgical navigation system using at least one of the following: infrared, sound, visual, magnetic, electromagnetic, and x-ray.

31. The method of claim 29, wherein the fiducial markers can be at least one of the following: a geometric shape, a sphere, a block, and a plate.

32. The method of claim 26, wherein the mount can be mounted to at least one of the following: a portion of a patient's body, a surgical instrument, a surgical item, an item associated with a surgery.

33. The method of claim 26, wherein the mount comprises at least one of the following: a pronged connector, a magnet, a threaded connector, an adhesive, and a bone screw.

34. The method of claim 26, wherein the surgical instrument comprises at least one of the following: a surgical implement, a surgical reference, a surgical trial, an implant, a cutting block, a reamer, a drill, a saw, an extramedullary rod, and an intramedullar rod.

35. A surgical method for locating a position of a joint replacement prosthesis associated with a navigational array using a computer-aided surgical navigation system, wherein the navigational array can be positioned for sensing by at least two sensors associated with the computer-aided surgical navigation system in order to determine position and orientation of the array by the system, the method comprising:
(a) providing a navigational array, wherein the navigational array comprises:
(i) a plurality of fiducial members adapted to be sensed by the computer-aided surgical navigation system;
(ii) a non-segmenting common point connecting at least some of the fiducial members, wherein at least one of the fiducial members is out of plane with the other three fiducial members, and wherein a position and orientation associated with the navigational array can be determined from sensing at least three of the fiducial members by a computer-aided surgical navigation system;
(iii) a mount adapted to support the navigational array adjacent to a joint replacement prosthesis; and
(iv) whereby the physical possibility for all of the fiducial members to be positioned coplanar to at least two sensors in the computer-aided surgical navigation system is precluded, and whereby when two of the fiducial members are positioned collinear to one of the sensors in the computer-aided surgical navigation system, no other of said members are positioned collinear to any other of said sensors;
(b) mounting the navigational array adjacent to a joint replacement prosthesis;
(c) sensing a portion of at least three of the fiducial members by a computer-aided surgical navigation system;
(d) determining a position associated with the joint replacement prosthesis based in part on sensing the portions of the at least three of the fiducial members; and
(e) mounting the joint replacement prosthesis to another corresponding joint replacement prosthesis for a joint replacement.

36. The method of claim 35, wherein at least three of the fiducial members are oriented in a flat, coplanar, Y-shaped configuration, and a fourth fiducial member is orthogonally oriented to each of the other three fiducial members.

37. The method of claim 35, wherein at least a portion of each fiducial member can be sensed by the computer-aided surgical navigation system using at least one of the following: infrared, sound, visual, magnetic, electromagnetic, and x-ray.

38. The method of claim 35, wherein each fiducial member comprises a respective fiducial marker.

39. The method of claim 38, wherein the fiducial markers can be sensed by a computer-aided surgical navigation system using at least one of the following: infrared, sound, visual, magnetic, electromagnetic, and x-ray.

40. The method of claim 38, wherein the fiducial markers can be at least one of the following: a geometric shape, a sphere, a block, and a plate.

41. The method of claim 35, wherein the mount comprises at least one of the following: a pronged connector, a magnet, a threaded connector, an adhesive, and a bone screw.

42. The method of claim 35, wherein the joint replacement prosthesis comprises at least one of the following: a tibial component, a femoral component.
